(19) United States
(12) Patent Application Publication
(10) Pub. No.: US 2024/0203283 A1
(43) Pub. Date: Jun. 20, 2024

(54) COGNITIVE FUNCTION TESTING DEVICE AND STORAGE MEDIUM

(71) Applicant: OMRON HEALTHCARE CO., LTD., Muko-shi (JP)

(72) Inventors: Miki OKUGAWA, Kyoto (JP); Yuka SUZUKI, Kyoto (JP); Kaoru ADACHI, Kyoto (JP)

(21) Appl. No.: 18/592,010

(22) Filed: Feb. 29, 2024

Related U.S. Application Data
(63) Continuation of application No. PCT/JP2023/004203, filed on Feb. 8, 2023.

(30) Foreign Application Priority Data
Jun. 20, 2022 (JP) 2022-098912

(51) Int. Cl. G09B 7/02; A61B 5/00; G16H 10/20

(52) U.S. Cl. CPC G09B 7/02; A61B 5/4088; G16H 10/20

(57) ABSTRACT

A cognitive function testing device, includes: a controller configured to an output process of outputting a first word to be memorized to an examinee, and an answer reception process of receiving, by causing a display device to display an answer image for receiving an answer for question words including the first word and a second word different from the first word sequentially, the answer from the examinee, the answer being as to whether the question word has been output in the output process. When the question word is switched in the answer reception process, the controller changes a display mode of the answer image.

### CROSS-REFERENCE TO RELATED APPLICATIONS

This application is a continuation of PCT application No. PCT/JP2023/004203, which was filed on Feb. 8, 2023 based on Japanese Patent Application No. 2022-098912 filed on Jun. 20, 2022, the contents of which are incorporated herein by way of reference.

### BACKGROUND

The present disclosure relates to a cognitive function testing device and a storage medium storing a cognitive function testing program.

In the related art, there is known a dementia diagnostic device that allows an examinee to memorize a plurality of words as memory words, and then displays the memory words and dummy words to allow the examinee to select the memory words from the words (JP2009-89800A).

According to the dementia diagnostic device of JP2009-89800A, after the memory words are sequentially displayed and memorized, one word among the words including the memory words and the dummy words is displayed on a display. In addition, a “YES” button to be selected when the word displayed on the display is present in the memory words, a “NO” button to be selected when the word is not present in the memory words, and an “UNKNOWN” button to be selected when the examinee is unsure of which one are displayed to allow the examinee to select one of the buttons.

However, JP2009-89800A does not describe how the three selection buttons displayed on the display are arranged on the display. Therefore, it is assumed that each of the three selection buttons is fixedly displayed at a predetermined display position. In this way, in a case where each of the selection buttons is fixed at a predetermined position, for example, when the number of questions increases, there is a possibility that the examinee continues to press the button at the same position from force of habit. In such a case, a memory function of the examinee may be unable to be correctly evaluated.

Aspects of the present disclosure relates to providing a cognitive function testing device and a storage medium storing a cognitive function testing program that are capable of improving test accuracy.

### SUMMARY

According to an aspect of the present disclosure, there is provided a cognitive function testing device, including:
a controller configured to
   an output process of outputting a first word to be memorized to an examinee, and
   an answer reception process of receiving, by causing a display device to display an answer image for receiving an answer for question words including the first word and a second word different from the first word sequentially, the answer from the examinee, the answer being as to whether the question word has been output in the output process, wherein
when the question word is switched in the answer reception process, the controller changes a display mode of the answer image.

### BRIEF DESCRIPTION OF DRAWINGS

FIG. 1 is a diagram showing an example of an information terminal to which a cognitive function testing device according to an embodiment is applied;

FIG. 2 is a diagram showing an example of a hardware configuration of the information terminal;

FIG. 3 is a flowchart showing an example of an overall process procedure of a cognitive function test;

FIG. 4 is a diagram showing an example of a test word displayed in an output process;

FIG. 5 is a diagram showing an example of an image displayed in a training process;

FIG. 6 is a diagram showing an example of an image displayed at the start of an answer reception process;

FIG. 7 is a diagram showing an example of an answer image displayed at the time of answering in the answer reception process;

FIG. 8 is a diagram showing an example of a touch operation at the time of answering in the answer reception process;

FIG. 9 is a diagram showing an example of an answer image displayed at the time of answering in the answer reception process;

FIG. 10 is a diagram showing a modified display mode 1 of the answer image;

FIG. 11 is a diagram showing a modified display mode 2 of the answer image;

FIGS. 12A and 12B are diagrams showing a modified display mode 3 of the answer image;

FIG. 13 is a diagram showing a modified display mode 4 of the answer image;

FIG. 14 is a diagram showing a modified display mode 5 of the answer image;

FIG. 15 is a diagram showing a modified display mode 6 of the answer image;

FIG. 16 is a diagram showing a modified display mode 7 of the answer image;

FIG. 17 is a diagram showing another modified display mode of the answer image;

FIG. 18 is a diagram showing another modified display mode of the answer image;

FIG. 19 is a diagram showing an example of an image displayed in a result output process; and

FIG. 20 is a diagram showing a modification of a controller.

### DESCRIPTION OF EMBODIMENTS

Hereinafter, an embodiment according to an aspect of the present disclosure will be described with reference to the drawings.

### § 1 Application Example

### <Information Terminal 10>

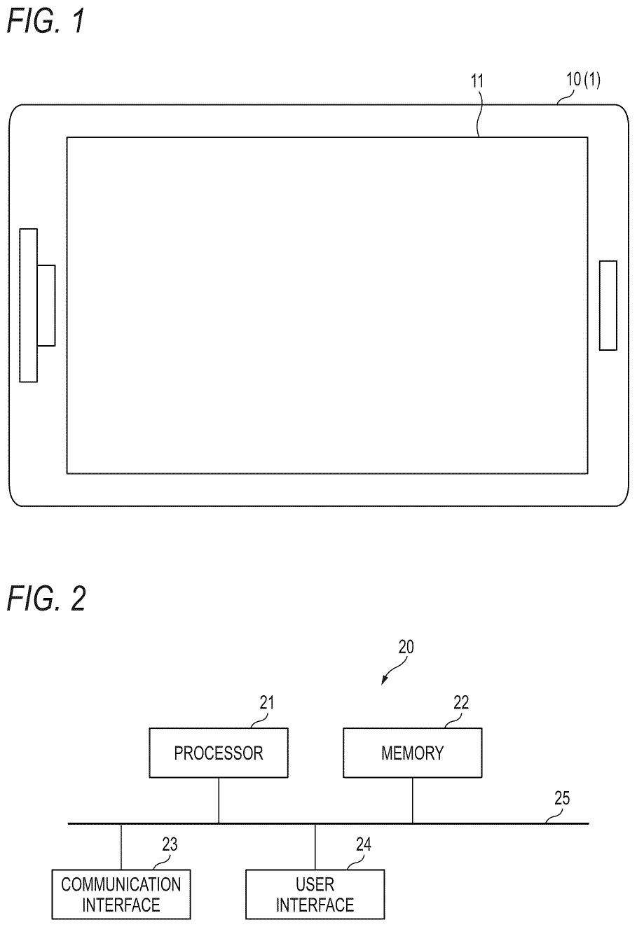

FIG. 1 is a diagram showing an example of an information terminal 10 on which a cognitive function testing device 1 according to the present embodiment is mounted. The information terminal 10 includes a display screen 11 on which test information for performing a cognitive function test is displayed. The information terminal 10 is, for example, a terminal such as a smartphone or a tablet. The display screen 11 also functions as a touch panel capable of inputting the test information. In addition, the information terminal 10 includes a speaker (not shown) that outputs the test information by voice.

### § 2 Configuration Example

### <Hardware Configuration of Cognitive Function Testing Device 1>

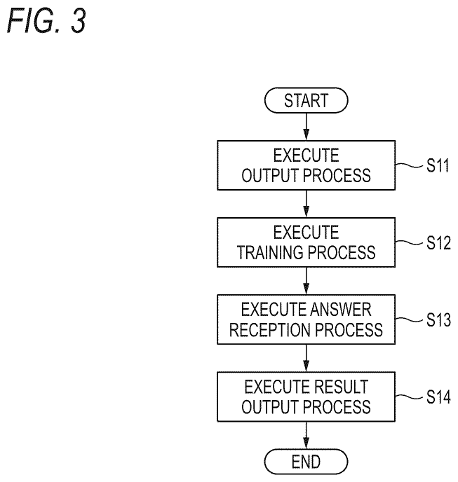

FIG. 2 is a diagram showing an example of a hardware configuration of the cognitive function testing device 1. The hardware of the cognitive function testing device 1 may be implemented by, for example, an information processing device 20 shown in FIG. 2. The information processing device 20 includes a processor 21, a memory 22, a communication interface 23, and a user interface 24. The processor 21, the memory 22, the communication interface 23, and the user interface 24 are connected by, for example, a bus 25.

The processor 21 is a circuit that performs a signal process, and is, for example, a central processing unit (CPU) that controls the entire information processing device 20. The processor 21 is an example of a controller of the present disclosure. The processor 21 may be implemented by another digital circuit such as a field programmable gate array (FPGA) or a digital signal processor (DSP). The processor 21 may be implemented by combining a plurality of digital circuits.

The memory 22 includes, for example, a main memory and an auxiliary memory. The main memory is, for example, a random access memory (RAM). The main memory is used as a work area of the processor 21.

The auxiliary memory is, for example, a nonvolatile memory such as a magnetic disk, an optical disk, or a flash memory. The auxiliary memory stores various programs for operating the information processing device 20. The program stored in the auxiliary memory is loaded into the main memory and executed by the processor 21.

The auxiliary memory may include a portable memory that is removable from the information processing device 20. Examples of the portable memory include a memory card such as a universal serial bus (USB) flash drive and a secure digital (SD) memory card, and an external hard disk drive.

The communication interface 23 is a communication interface that performs network communication with the outside (for example, an external server) of the information processing device 20. The communication interface 23 is controlled by the processor 21.

The user interface 24 includes, for example, an input device that receives an operation input from an examinee who receives the cognitive function test and an output device that outputs information to the examinee. The input device may be implemented by, for example, a touch panel. The output device may be implemented by, for example, a display or a speaker. The user interface 24 is controlled by the processor 21.

For example, in the cognitive function test, the processor 21 executes an output process of outputting a “test word” to be memorized to the examinee, an answer reception process of receiving from the examinee an answer as to whether a word is the test word output in the output process, and a result output process of outputting a result of the cognitive function test. The processor 21 may execute, for example, a training process between the output process and the answer reception process.

### <Output Process>

The processor 21 reproduces a plurality of test words to the examinee via the display screen 11 and the speaker of the information terminal 10. For example, by sequentially displaying the test words as images or texts on the display screen 11, the processor 21 causes the examinee to memorize the displayed test words. In addition, for example, the processor 21 may output the test words as a voice via the speaker of the information terminal 10 in combination with the image display or the text display. The test words to be reproduced are stored in the memory 22 as image information, voice information, and text information. The test word is an example of a first word and a second word.

### <Answer Reception Process>

The processor 21 sequentially displays, on the display screen 11, a plurality of “question words” including the plurality of test words that have been output to the examinee in the output process and a plurality of “non-test words” not included in the plurality of test words. Also in this case, the display of the question words on the display screen 11 may be the image display or the text display. In addition, the voice output may be combined with the image display or the text display. In addition to displaying the question word, the processor 21 also displays, on the display screen 11, an “answer image” for answering whether the displayed question word is the test word that has been output in the output process. Then, the processor 21 receives an answer to the displayed question word, for example, based on a selection by a touch operation of the answer image from the examinee.

When switching the question word to be displayed on the display screen 11 to another question word, the processor 21 changes a display mode of the answer image to be displayed together with the question word. For example, when switching the question word, the processor 21 changes a display arrangement of the answer image. For example, when switching the question word, the processor 21 may change a display interval between the answer images, a display size of the answer image, a display shape of the answer image, a movement of the answer image, a design of the answer image, or a display timing of the answer image.

### <Result Output Process>

The processor 21 displays, on the display screen 11, a correct/incorrect answer determination result for each question word received from the examinee in the answer reception process. For example, the processor 21 displays, on the display screen 11, the number of correct answers for the number of questions as a test score of the examinee.

### § 3 Operation Example

### <Operation of Cognitive Function Testing Device>

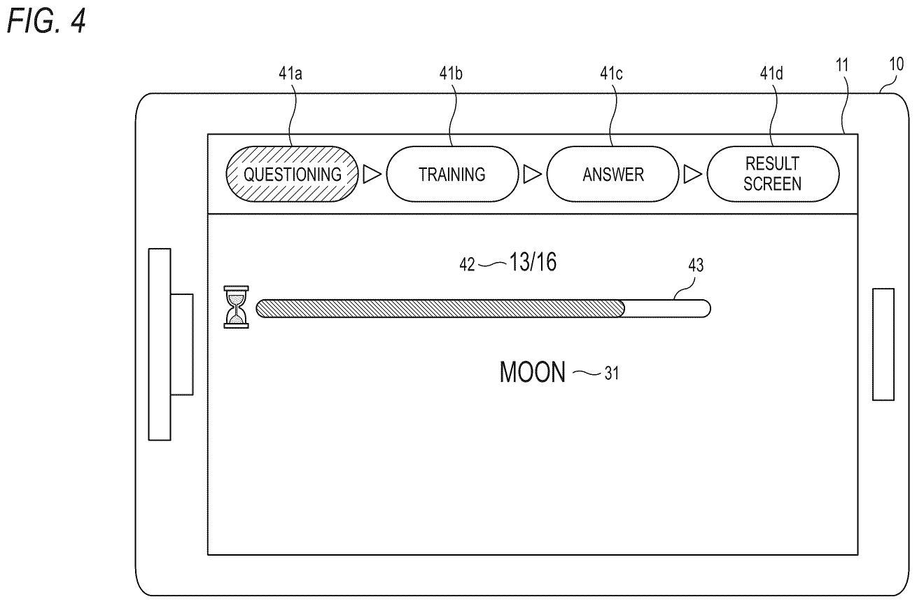

Next, an operation example of the cognitive function test of the cognitive function testing device 1 will be described with reference to FIGS. 3 to 19.

FIG. 3 is a flowchart showing an overall process procedure of the cognitive function test. As shown in FIG. 3, the processor 21 of the cognitive function testing device 1 performs the cognitive function test in the order of the output process in step S11, the training process in step S12, the answer reception process in step S13, and the result output process in step S14. The cognitive function test is started by a touch operation on the display screen 11 of the information terminal 10.

### <Output Process in Step S11>

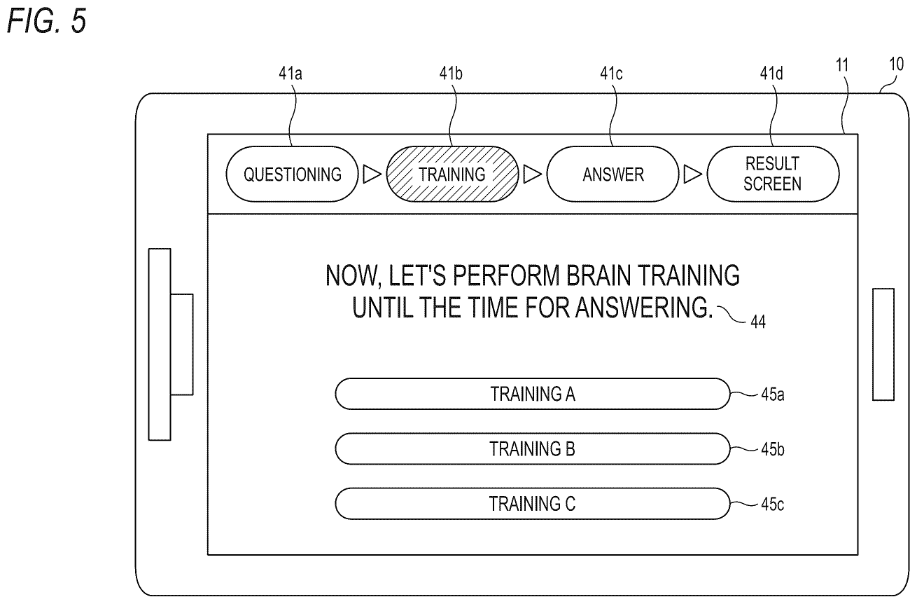

When the output process starts, the processor 21 announces, for example, “PLEASE MEMORIZE WORDS TO BE DISPLAYED ON SCREEN. THERE ARE 16 QUESTIONS. NOW, LET'S START.”. Then, for example, as shown in FIG. 4, the processor 21 sequentially displays test words 31 to be memorized by the examinee on the display screen 11 word by word. The test words 31 are displayed, for example, from the first question to the 16th question. A display time of each test word may be set freely, for example, to about 5 seconds. In the example shown in the drawing, the test word 31 such as “moon” is displayed in characters. The processor 21 displays, on the display screen 11, a questioning mark 41a for notifying that a current test process is the “output process” among the “output process”, the “training process”, the “answering process”, and the “result process”. The processor 21 displays, on the display screen 11, a question number mark 42 for notifying that a current question is, for example, the 13th question among the 16 questions, and an elapsed time bar 43 indicating an elapsed time of the output process.

### <Training Process in Step S12>

When the output process in step S11 ends, the processor 21 executes the training process of reproducing content different from the cognitive function test in order to apply a load on the cognition of the examinee. That is, the training process is a training that is performed so that the examinee who memorizes the word to be memorized in the output process is not asked to answer the memorized word immediately, but is asked to answer after a little time.

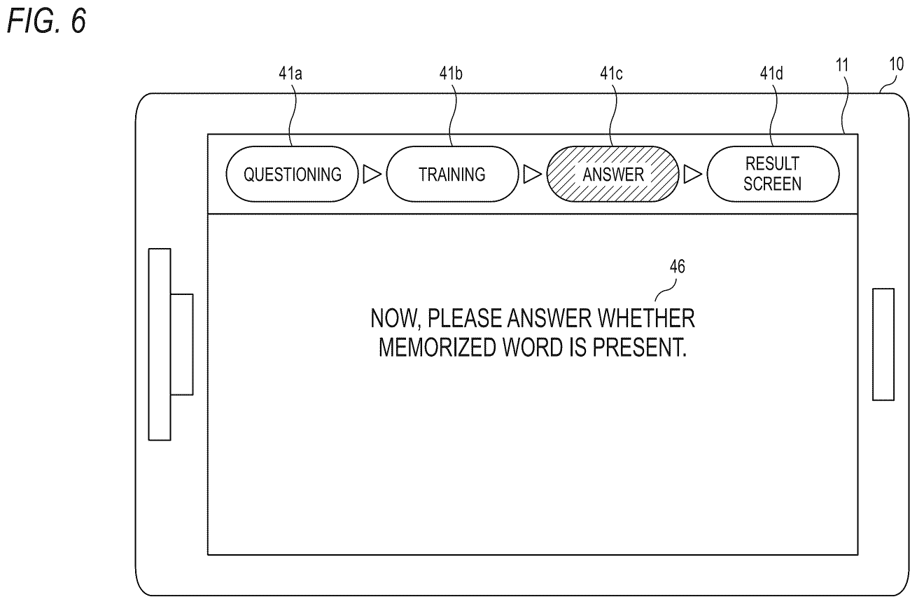

When the process shifts from the output process to the training process, as shown in FIG. 5, the processor 21 displays a training mark 41b for notifying that the current process is the training process, and displays a comment 44 such as “NOW, LET'S PERFORM BRAIN TRAINING UNTIL THE TIME FOR ANSWERING”. The training is configured such that the examinee may freely select one of trainings A, B, and C, for example. The processor 21 displays selection buttons 45a to 45c for selecting a type of training.

### <Answer Reception Process in Step S13>

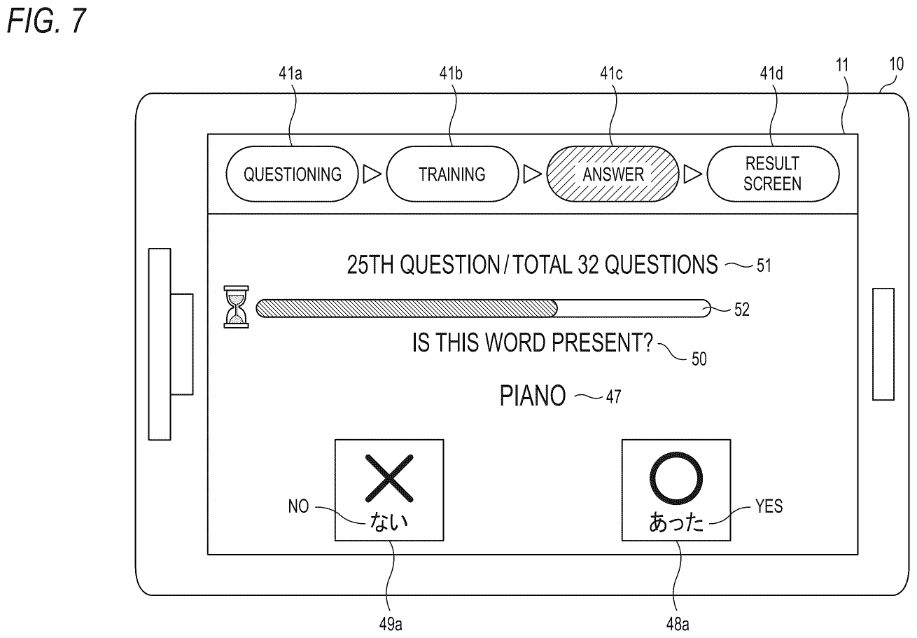

When the training process in step S12 ends, the processor 21 sequentially displays a plurality of words on the display screen 11, and executes the answer reception process of causing the examinee to answer whether the displayed word is the test word questioned in the output process. For example, as shown in FIG. 6, the processor 21 displays an answer mark 41c for notifying that the current process is the answer reception process, and displays a comment 46 such as “NOW, PLEASE ANSWER WHETHER MEMORIZED WORD IS PRESENT”. Accordingly, a process of receiving an answer is started.

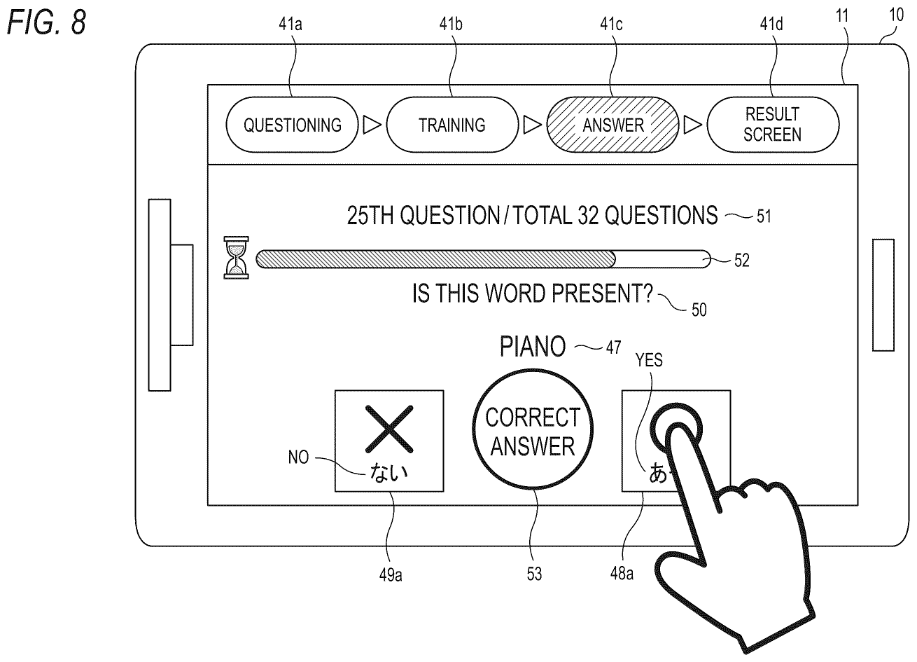

FIG. 7 is an example of a question image for one question displayed on the display screen 11 at the time of answering each question. As shown in FIG. 7, the processor 21 sequentially displays, on the display screen 11, question words 47 for which the examinee is asked to answer whether the words are present in the test words. The question words 47 are displayed, for example, from the first question to the 32nd question. In the example shown in the drawing, the question word 47 such as “piano” is displayed in characters.

The processor 21 displays, together with the question word 47, an answer button on which a positive answer image 48a for answering that the question word 47 has been output as the test word is displayed, and an answer button on which a negative answer image 49a for answering that the question word 47 has not been output as the test word is displayed. The processor 21 displays a question comment 50 such as “IS THIS WORD PRESENT?”.

Further, the processor 21 displays a question number mark 51 for notifying that the current question is, for example, the 25th question among the 32 questions, and an elapsed time bar 52 indicating an elapsed time for answering the 25th question. An answer time for each question may be set freely, for example to about 5 seconds. The positive answer image 48a is an example of a first answer image. The negative answer image 49a is an example of a second answer image.

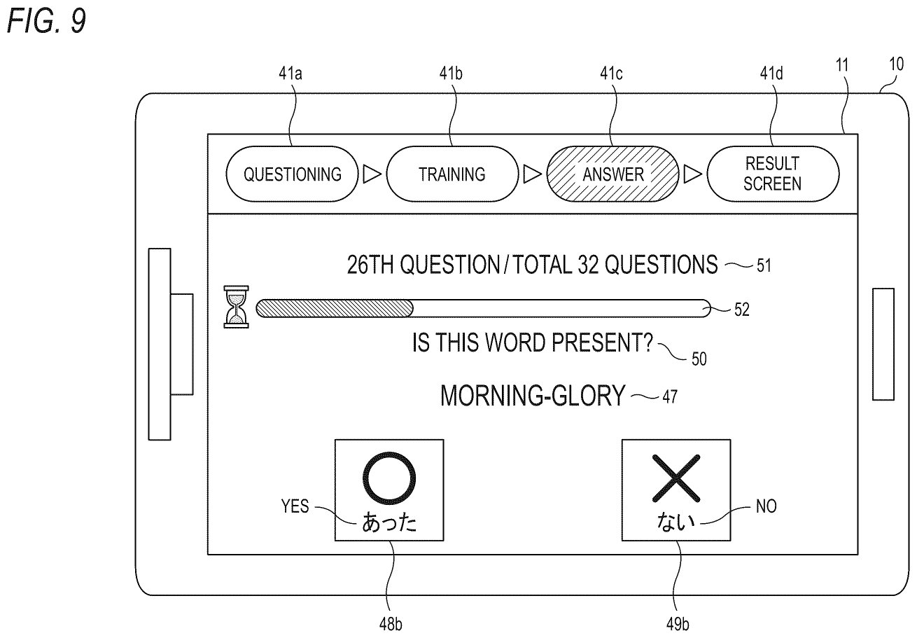

In the question image shown in FIG. 7, the examinee performs a touch operation on the positive answer image 48a when the examinee memorizes that “piano” is present in the test words, and performs a touch operation on the negative answer image 49a when the examinee memorizes that “piano” is not present in the test words. FIG. 8 is a diagram showing a state in which the examinee performs the touch operation on the positive answer image 48a. Then, when “piano” is present in the test words in response to the answer of the examinee, a correct answer mark 53 is displayed as shown in FIG. 8. On the other hand, when “piano” is not present in the test words, an incorrect answer mark (for example, a “x” image and a character “incorrect answer”) is displayed.

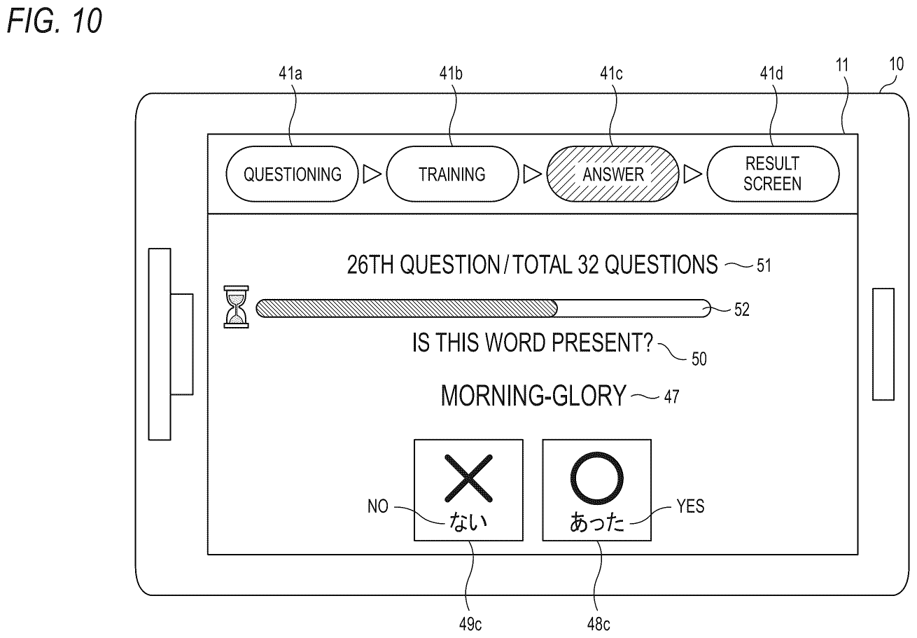

As described in FIGS. 7 and 8, when the answer to the 25th question ends, the processor 21 displays a question image for the 26th question on the display screen 11. FIG. 9 is an example of the question image displayed on the display screen 11 at the time of answering the 26th question. As shown in FIG. 9, the processor 21 displays “morning-glory” in characters as the question word 47 in the question image for the 26th question.

In addition, the processor 21 displays, together with “morning-glory” (question word 47), a positive answer image 48b for answering that “morning-glory” has been output as the test word and a negative answer image 49b for answering that “morning-glory” has not been output as the test word. At this time, the processor 21 changes a display arrangement of the positive answer image 48b and the negative answer image 49b as the answer images for the 26th question from a display arrangement of the positive answer image 48a and the negative answer image 49a as the answer images for the 25th question. Specifically, in the question image for the 25th question shown in FIG. 7, the processor 21 displays the negative answer image 49a on a right side of the display screen 11 and displays the positive answer image 48a on a left side, whereas in the question image for the 26th question shown in FIG. 9, the processor 21 displays the positive answer image 48b on the right side of the display screen 11 and displays the negative answer image 49b on the left side. The question number mark 51 indicates that the question is the 26th question among the 32 questions. Display contents of the question comment 50 and the elapsed time bar 52 are the same as those of the 25th question.

In the question image shown in FIG. 9, a process after the examinee answers by a touch operation is the same as the process for answering the 25th question.

### <Modified Display Mode of Answer Image>

A display mode of the answer image for the 26th question “morning-glory” described above changes the display arrangement as compared with a display mode of the answer image for the 25th question “piano”, but the present disclosure is not limited thereto.

### <Modified Display Mode 1>

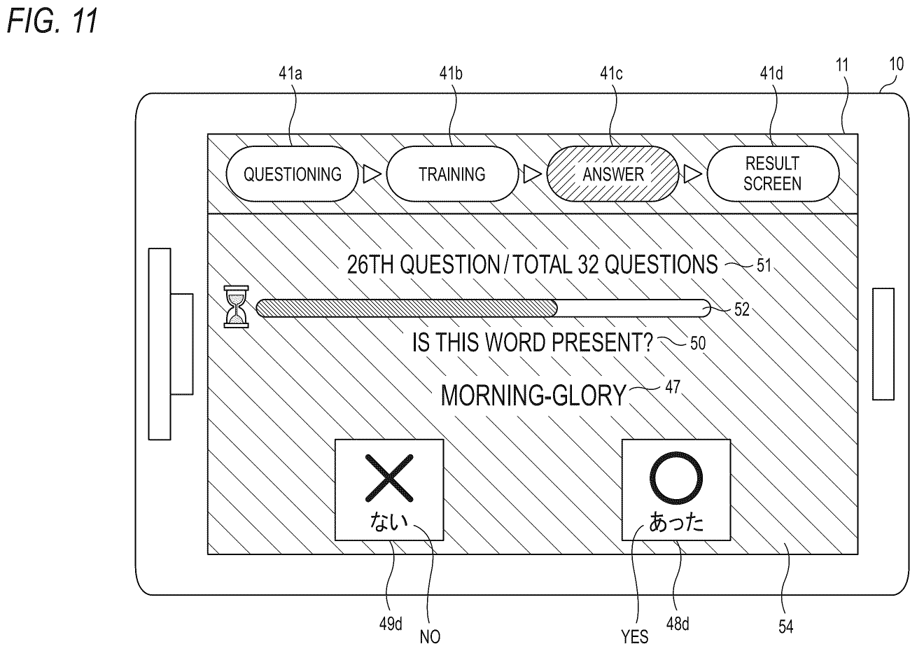

FIG. 10 is a diagram showing a modified display mode 1 of the answer image for the 26th question “morning-glory”. As shown in FIG. 10, the processor 21 may change a display interval between a positive answer image 48c and a negative answer image 49c as the answer images for the 26th question “morning-glory” from a display interval between the positive answer image 48a and the negative answer image 49a as the answer images for the 25th question “piano”. Specifically, the processor 21 may make the display interval between the positive answer image 48c and the negative answer image 49c displayed on the display screen 11 in the question image for the 26th question “morning-glory” shown in FIG. 10 smaller than the display interval between the positive answer image 48a and the negative answer image 49a displayed on the display screen 11 in the question image for the 25th question “piano” shown in FIG. 7.

### <Modified Display Mode 2>

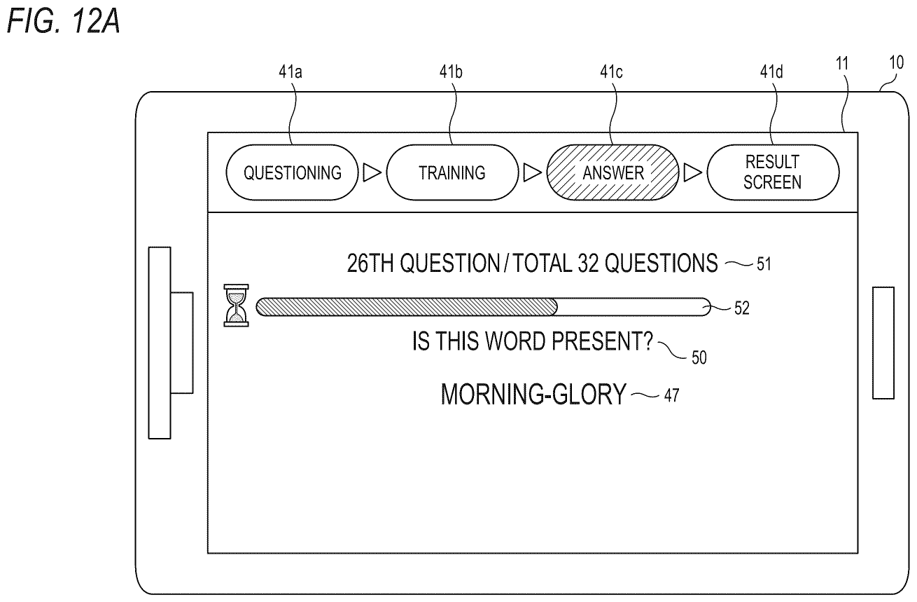

FIG. 11 is a diagram showing a modified display mode 2 of the answer image for the 26th question “morning-glory”. As shown in FIG. 11, the processor 21 may change a design of the answer image for the 26th question “morning-glory” from a design of the answer image for the 25th question “piano”. Specifically, instead of displaying, in colorless, a background image of the positive answer image 48a and the negative answer image 49a as the answer images for the 25th question “piano” shown in FIG. 7, the processor 21 may display, in color, a background image 54 of a positive answer image 48d and a negative answer image 49d as the answer images for the 26th question “morning-glory” shown in FIG. 11. However, the “design of the answer image” may be a design of information (text information on “piano”, “morning-glory”, or the like) to be conveyed to the examinee or information (color, font, or the like of the background image of the answer image) related to appearance (design), other than designs such as “∘”, “x”, “YES”, and “NO” that are elements included in the answer image. Alternatively, the design may be a design of an appearance of the answer image.

### <Modified Display Mode 3>

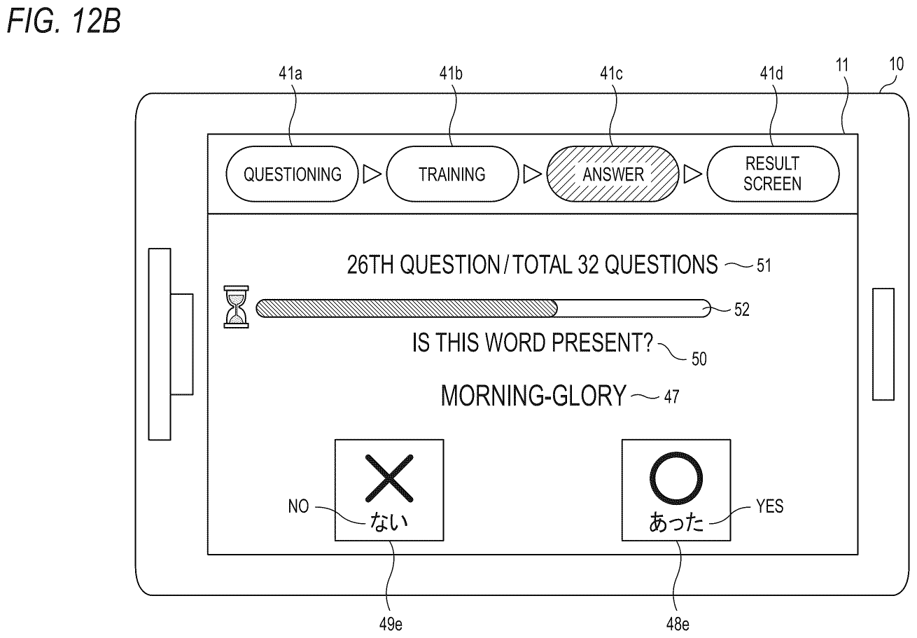

FIGS. 12A and 12B are diagrams showing a modified display mode 3 of the answer image for the 26th question “morning-glory”. As shown in FIGS. 12A and 12B, the processor 21 may change a display timing of a positive answer image 48e and a negative answer image 49e as the answer images for the 26th question “morning-glory” from a display timing of the positive answer image 48a and the negative answer image 49a as the answer images for the 25th question “piano”. Specifically, instead of simultaneously displaying “piano” (question word 47) and the positive answer image 48a and the negative answer image 49a on the display screen 11 in the question image for the 25th question “piano” shown in FIG. 7, the processor 21 may first display “morning-glory” (question word 47) on the display screen 11 in the question image for the 26th question “morning-glory” as shown in FIG. 12A and display the positive answer image 48c and the negative answer image 49c a little later (for example, after 0.5 seconds) as shown in FIG. 12B.

### <Modified Display Mode 4>

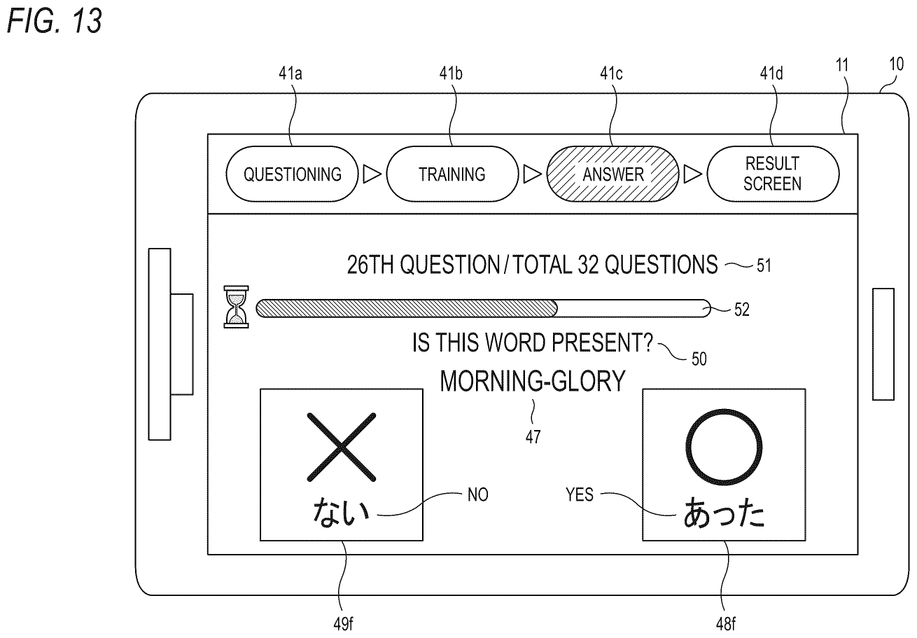

FIG. 13 is a diagram showing a modified display mode 4 of the answer image for the 26th question “morning-glory”. As shown in FIG. 13, the processor 21 may change a display size of a positive answer image 48f and a negative answer image 49f as the answer images for the 26th question “morning-glory” from a display size of the positive answer image 48a and the negative answer image 49a as the answer images for the 25th question “piano”. Specifically, the processor 21 may make the display size of the positive answer image 48f and the negative answer image 49f displayed on the display screen 11 in the question image for the 26th question “morning-glory” shown in FIG. 13 larger than the display size of the positive answer image 48a and the negative answer image 49a displayed on the display screen 11 in the question image for the 25th question “piano” shown in FIG. 7.

### <Modified Display Mode 5>

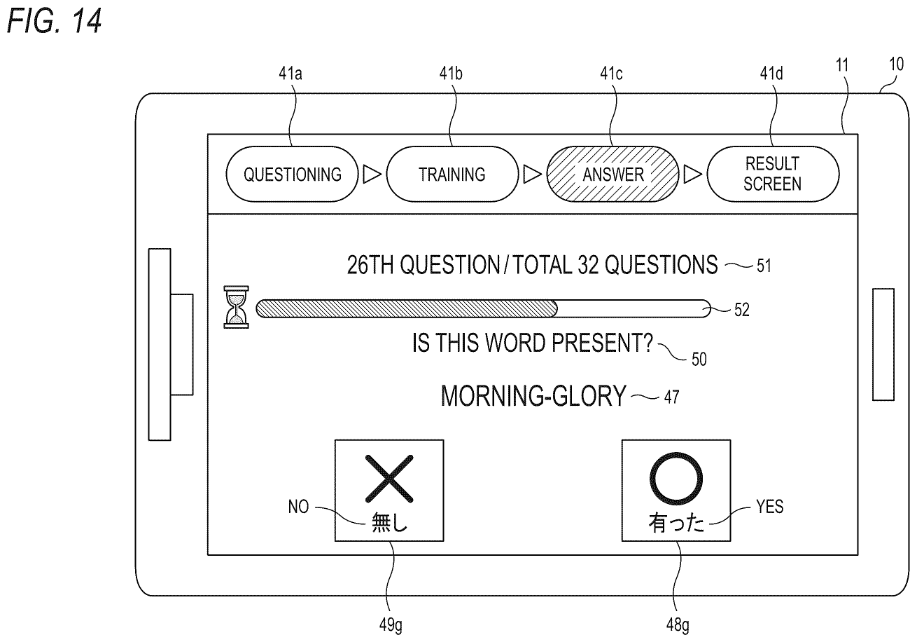

FIG. 14 is a diagram showing a modified display mode 5 of the answer image for the 26th question “morning-glory”. As shown in FIG. 14, the processor 21 may change a display character type of a positive answer image 48g and a negative answer image 49g as the answer images for the 26th question “morning-glory” from a display character type of the positive answer image 48a and the negative answer image 49a as the answer images for the 25th question “piano”. Specifically, instead of displaying, in hiragana, display characters of the positive answer image 48a and the negative answer image 49a displayed on the display screen 11 in the question image for the 25th question “piano” shown in FIG. 7, the processor 21 may display, in a character string including Kanji characters, display characters of the positive answer image 48g and the negative answer image 49g displayed on the display screen 11 in the question image for the 26th question “morning-glory” shown in FIG. 14.

### <Modified Display Mode 6>

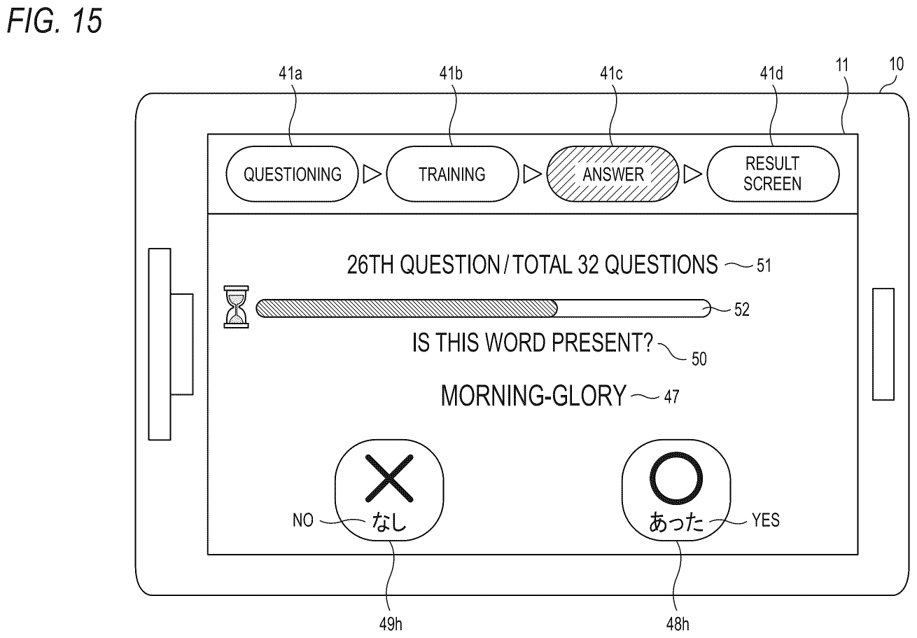

FIG. 15 is a diagram showing a modified display mode 6 of the answer image for the 26th question “morning-glory”. As shown in FIG. 15, the processor 21 may change the shape of a positive answer image 48h and a negative answer image 49h as the answer images for the 26th question “morning-glory” from the shape of the positive answer image 48a and the negative answer image 49a as the answer images for the 25th question “piano”. Specifically, instead of displaying, in a rectangular shape, the answer button on which the positive answer image 48a is displayed and the answer button on which the negative answer image 49a is displayed in the question image for the 25th question “piano” shown in FIG. 7, the processor 21 may display, in a rectangular shape with rounded corners, a substantially circular shape, or the like, an answer button on which the positive answer image 48h is displayed and an answer button on which the negative answer image 49h is displayed in the question image for the 26th question “morning-glory” shown in FIG. 15.

### <Modified Display Mode 7>

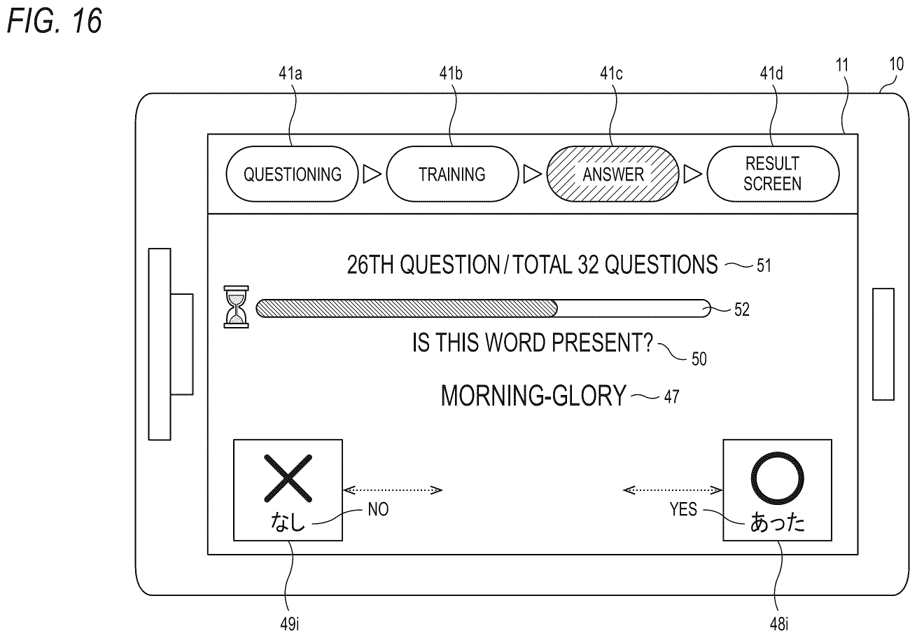

FIG. 16 is a diagram showing a modified display mode 7 of the answer image for the 26th question “morning-glory”. As shown in FIG. 16, the processor 21 may move display positions of a positive answer image 48i and a negative answer image 49i as the answer images for the 26th question “morning-glory” on the display screen 11. Specifically, the processor 21 may move the positive answer image 48i and the negative answer image 49i back and forth in opposite directions in a left-right direction on the display screen 11, and display the images while moving the images closer to or away from each other. A moving direction of the answer image is not limited to the left-right direction.

### <Other Modified Display Modes>

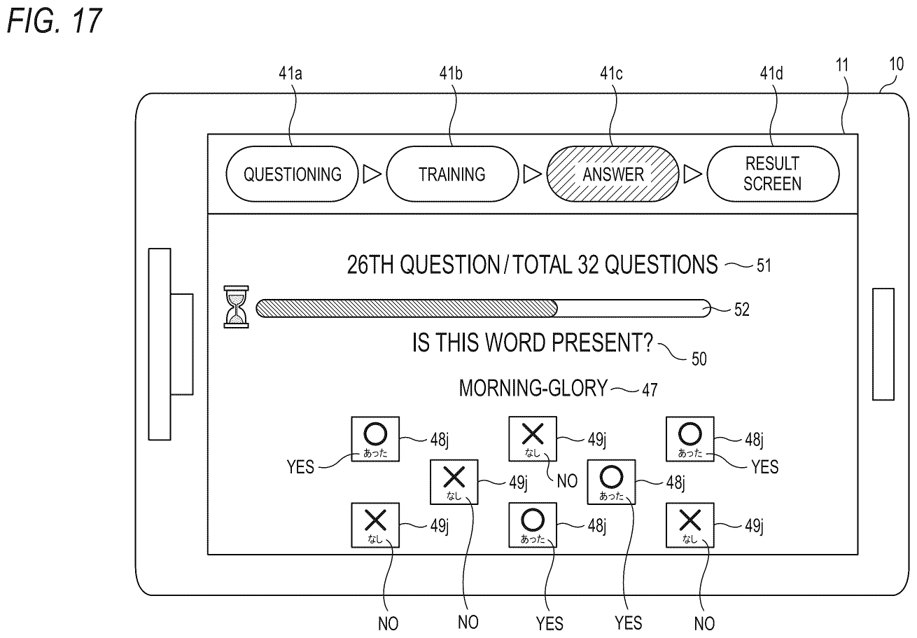

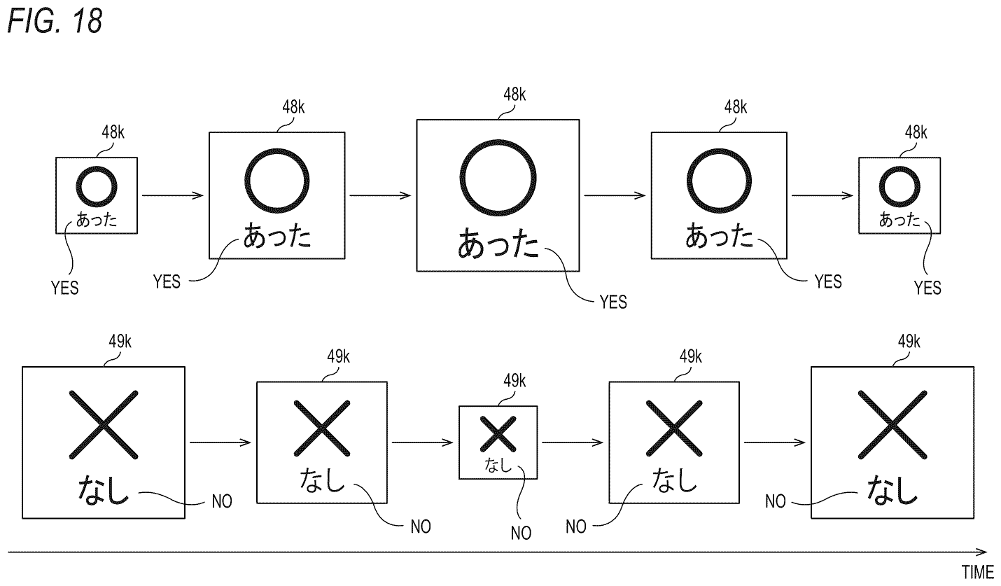

For example, as shown in FIG. 17, the processor 21 may display a plurality of positive answer images 48j and a plurality of negative answer images 49j as the answer images on the display screen 11. In addition, for example, in the example of FIG. 9 and the like, the processor 21 may display a positive answer image 48k and a negative answer image 49k as the answer images while increasing or decreasing a display size over time as shown in FIG. 18.

### <Result Output Process in Step S14>

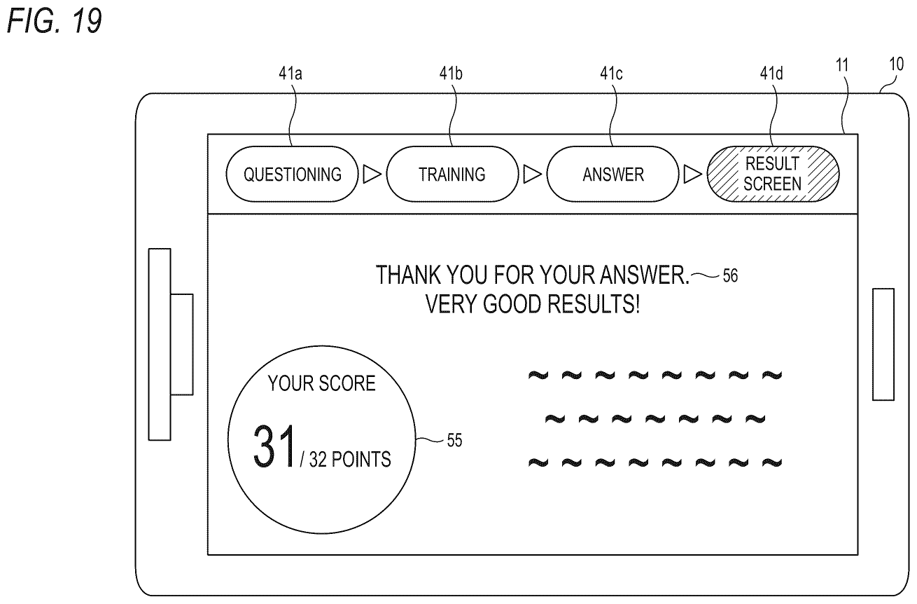

When the answer reception process of step S13 ends, the processor 21 executes the result output process for notifying a test result. When the process shifts from the answer reception process to the result output process, as shown in FIG. 19, the processor 21 displays a result screen mark 41d for notifying that the current process is the result output process, and displays a test result 55 indicating that 31 questions of the 32 questions were answered correctly, and a comment 56 such as “THANK YOU FOR YOUR ANSWER. VERY GOOD RESULTS!”. In this example, a case has been described in which, among the words (including dummy words) output in the answer reception process, the number of correct answers to determine whether the word is the test word questioned in the output process is output as a test result (for example, the number of correct answers in 32 questions), but the processor 21 may output, as the test result, the number of words (for example, the number of correct answers in 16 questions) that may be answered as the test word when the word is output in the answer reception process among the test words (not including dummy words) questioned in the output process.

As described above, when the question word to be questioned to the information terminal 10 is switched, the cognitive function testing device 1 changes the display mode of the answer image (positive answer image and negative answer image) for answering the question word. Therefore, a feeling of tension of the examinee is enhanced by the changed display mode of the answer image, and it is possible to prevent the examinee from performing a touch operation on the answer image from force of habit when answering a plurality of questions sequentially displayed on the information terminal 10, and to improve the test accuracy.

For example, when the question word to be questioned to the information terminal 10 is switched, as shown in FIG. 9, the cognitive function testing device 1 changes the display arrangement of the positive answer image 48b and the negative answer image 49b from the display arrangement of the positive answer image 48a and the negative answer image 49a (FIG. 7) and perform the reverse. Therefore, it is possible to prevent the examinee from performing the touch operation on the answer image from force of habit for the plurality of questions sequentially displayed.

For example, when the question word to be questioned to the information terminal 10 is switched, as shown in FIG. 10, the cognitive function testing device 1 changes the display interval between the positive answer image 48c and the negative answer image 49c to be smaller than the display interval between the positive answer image 48a and the negative answer image 49a (FIG. 7). Therefore, it is possible to prevent the examinee from performing the touch operation on the answer image from force of habit for the plurality of questions sequentially displayed.

For example, when the question word to be questioned to the information terminal 10 is switched, as shown in FIG. 11, the cognitive function testing device 1 changes the design to be different from the colorless background image of the positive answer image 48a and the negative answer image 49a (FIG. 7) by coloring the background image 54 of the positive answer image 48d and the negative answer image 49d. Therefore, it is possible to prevent the examinee from performing the touch operation on the answer image from force of habit for the plurality of questions sequentially displayed.

For example, when the question word to be questioned to the information terminal 10 is switched, as shown in FIGS. 12A and 12B, the cognitive function testing device 1 delays the display timing of the positive answer image 48e and the negative answer image 49e a little later than the display timing of the answer image “morning-glory”, so as to be different from a case where the positive answer image 48a and the negative answer image 49a as the answer images and the question word “piano” are displayed at the same time (FIG. 7). Therefore, it is possible to prevent the examinee from performing the touch operation on the answer image from force of habit for the plurality of questions sequentially displayed.

For example, when the question word questioned on the information terminal 10 is switched, as shown in FIG. 13, the cognitive function testing device 1 changes the display size of the positive answer image 48f and the negative answer image 49f to be larger than the display size of the positive answer image 48a and the negative answer image 49a (FIG. 7). Therefore, it is possible to prevent the examinee from performing the touch operation on the answer image from force of habit for the plurality of questions sequentially displayed.

For example, when the question word questioned on the information terminal 10 is switched, as shown in FIG. 14, the cognitive function testing device 1 sets the display character type of the positive answer image 48g and the negative answer image 49g to Kanji characters, so as to be different from the hiragana display of the positive answer image 48a and the negative answer image 49a (FIG. 7). Therefore, it is possible to prevent the examinee from performing the touch operation on the answer image from force of habit for the plurality of questions sequentially displayed.

For example, when the question word questioned on the information terminal 10 is switched, as shown in FIG. 15, the cognitive function testing device 1 makes the display shape of the positive answer image 48h and the negative answer image 49h circular, so as to be different from the rectangular shape of the positive answer image 48a and the negative answer image 49a (FIG. 7). Therefore, it is possible to prevent the examinee from performing the touch operation on the answer image from force of habit for the plurality of questions sequentially displayed.

For example, when the question word questioned on the information terminal 10 is switched, as shown in FIG. 16, the cognitive function testing device 1 moves the display positions of the positive answer image 48i and the negative answer image 49i on the display screen 11, so as to be different from a case where the display positions of the positive answer image 48a and the negative answer image 49a are not moved (FIG. 7). Therefore, it is possible to prevent the examinee from performing the touch operation on the answer image from force of habit for the plurality of questions sequentially displayed.

### § 4 Modifications

Although the embodiment of the present disclosure has been described in detail above, the above description is merely an example of the present disclosure in all respects. Various improvements and modifications may be made without departing from the scope of the present disclosure. For example, the following changes are possible. In the following description, the same components as those of the above embodiment are denoted by the same reference signs, and the description of the same points as in the above embodiment is omitted as appropriate. The following modifications may be combined as appropriate.

### <[Modification 1>

### <Modification of Processor (Controller) 21>

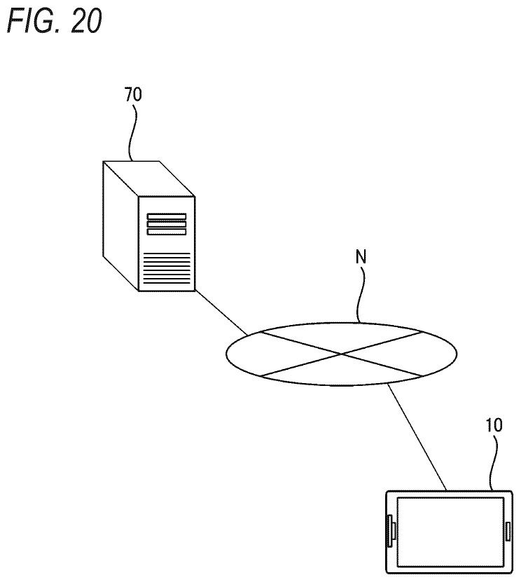

A modification of the processor (controller) 21 will be described with reference to FIG. 20. In the above embodiment, a case where the cognitive function testing device 1 is implemented by the processor 21 mounted on the information processing device 20 of the information terminal 10 has been described, but the present disclosure is not limited thereto. For example, as shown in FIG. 20, the cognitive function testing device 1 may be implemented by an external server 60 connectable to the information terminal 10 via a wide area network N such as the Internet. That is, the cognitive function test may be provided as a cognitive function test page that may be viewed using a web browser from the information terminal 10.

### § 5 Program

The cognitive function testing method described in the above embodiment may be implemented by executing a cognitive function testing program prepared in advance on a computer. The cognitive function testing program is stored in a computer-readable storage medium and is executed by being read from the storage medium. The cognitive function testing program may be provided in a form stored in a non-transitory storage medium such as a flash memory, or may be provided via a network such as the Internet. The computer that executes the cognitive function testing program may be provided in the cognitive function testing device, may be provided in an electronic device such as a smartphone, a tablet terminal, or a personal computer that may communicate with the cognitive function testing device, or may be provided in a server device that may communicate with the cognitive function testing device and the electronic device.

The present disclosure includes following configurations.

(1) A cognitive function testing device, including:
a controller configured to execute
an output process of outputting a first word to be memorized to an examinee, and
an answer reception process of receiving, by causing a display device to display an answer image for receiving an answer for question words including the first word and a second word different from the first word sequentially, the answer from the examinee, the answer being as to whether the question word has been output in the output process, in which
when the question word is switched in the answer reception process, the controller changes a display mode of the answer image.

According to (1), when the question word questioned on the display device is switched, the display mode of the answer image for answering the question word is changed, and thus it is possible to prevent the examinee from answering the questions sequentially displayed from force of habit, and to improve test accuracy.

(2) The cognitive function testing device according to (1), in which
the answer image includes a first answer image for answering that the question word has been output in the output process and a second answer image for answering that the question word has not been output in the output process.

According to (2), the answer may be made by selecting either the first answer image or the second answer image, and thus the examinee may easily answer.

(3) The cognitive function testing device according to (2), in which
when the question word is switched in the answer reception process, the controller changes a display arrangement of the first answer image and the second answer image.

According to (3), when the question word questioned on the display device is switched, the display arrangement of the answer image is changed, and thus it is possible to prevent the examinee from answering the plurality of questions from force of habit.

(4) The cognitive function testing device according to (2) or (3), in which
when the question word is switched in the answer reception process, the controller changes a display interval between the first answer image and the second answer image.

According to (4), when the question word questioned on the display device is switched, the display interval between the answer images is changed, and thus it is possible to prevent the examinee from answering the plurality of questions from force of habit.

(5) The cognitive function testing device according to any one of (1) to (4), in which
when the question word is switched in the answer reception process, the controller changes a size of the answer image.

According to (5), when the question word questioned on the display device is switched, the display size of the answer image is changed, and thus it is possible to prevent the examinee from answering the plurality of questions from force of habit.

(6) The cognitive function testing device according to any one of (1) to (5), in which
when the question word is switched in the answer reception process, the controller changes a shape of the answer image.

According to (6), when the question word questioned on the display device is switched, the display shape of the answer image is changed, and thus it is possible to prevent the examinee from answering the plurality of questions from force of habit.

(7) The cognitive function testing device according to any one of (1) to (6), in which
the controller changes a movement of the answer image when the question word is switched in the answer reception process.

According to (7), when the question word questioned on the display device is switched, the movement of the answer image to be displayed is changed, and thus it is possible to prevent the examinee from answering the plurality of questions from force of habit.

(8) The cognitive function testing device according to any one of (1) to (7), in which
when the question word is switched in the answer reception process, the controller changes a design of the answer image.

According to (8), when the question word questioned on the display device is switched, the display design of the answer image is changed, and thus it is possible to prevent the examinee from answering the plurality of questions from force of habit.

(9) The cognitive function testing device according to any one of (1) to (8), in which
when the question word is switched in the answer reception process, the controller changes a display timing of the answer image.

According to (9), when the question word questioned on the display device is switched, the display timing of the answer image is changed, and thus it is possible to prevent the examinee from answering the plurality of questions from force of habit.

(10) The cognitive function testing device according to any one of (1) to (9), in which
the controller is configured to execute a training process between the output process and the answer reception process to reproduce content that applies a load on cognition of the examinee.

According to (10), the training process is executed between the questioning process and the answer reception process, and thus the test accuracy may be improved. The training process of applying a load on the cognition is a process of reproducing content for causing the examinee to perform an active task. The content for causing the examinee to perform the active task is, for example, content such as a questionnaire, a quiz, or a game.

(11) A non-transitory computer-readable storage medium storing a cognitive function testing program for causing a processor of a cognitive function testing device to execute
an output process of outputting a first word to be memorized to an examinee, and
an answer reception process of receiving, by causing a display device to display an answer image for receiving an answer for question words including the first word and a second word different from the first word sequentially, the answer from the examinee, the answer being as to whether the question word has been output in the output process, in which
the answer reception process includes a process of changing a display mode of the answer image when the question word is switched.

According to (11), when the question word questioned on the display device is switched, the display mode of the answer image for answering the question word is changed, and thus it is possible to prevent the examinee from answering the plurality of questions sequentially displayed from force of habit, and to improve test accuracy.

According to aspects of the present disclosure, it is possible to provide the cognitive function testing device and the storage medium storing the cognitive function testing program that are capable of improving the test accuracy.

Although various embodiments have been described above, it goes without saying that the present invention is not limited to these examples. It is apparent to those skilled in the art that various changes or modifications may be conceived within the scope described in the claims, and it is understood that the changes or modifications naturally fall within the technical scope of the present invention. In addition, the components described in the above embodiment may be freely combined without departing from the spirit of the invention.

What is claimed is:

1. A cognitive function testing device, comprising:
a controller configured to
an output process of outputting a first word to be memorized to an examinee, and
an answer reception process of receiving, by causing a display device to display an answer image for receiving an answer for question words including the first word and a second word different from the first word sequentially, the answer from the examinee, the answer being as to whether the question word has been output in the output process, wherein
when the question word is switched in the answer reception process, the controller changes a display mode of the answer image.

2. The cognitive function testing device according to claim 1, wherein
the answer image includes a first answer image for answering that the question word has been output in the output process and a second answer image for answering that the question word has not been output in the output process.

3. The cognitive function testing device according to claim 2, wherein
when the question word is switched in the answer reception process, the controller changes a display arrangement of the first answer image and the second answer image.

4. The cognitive function testing device according to claim 2, wherein
when the question word is switched in the answer reception process, the controller changes a display interval between the first answer image and the second answer image.

5. The cognitive function testing device according to claim 1, wherein
when the question word is switched in the answer reception process, the controller changes a size of the answer image.

6. The cognitive function testing device according to claim 1, wherein
when the question word is switched in the answer reception process, the controller changes a shape of the answer image.

7. The cognitive function testing device according to claim 1, wherein
when the question word is switched in the answer reception process, the controller changes a movement of the answer image.

8. The cognitive function testing device according to claim 1, wherein
when the question word is switched in the answer reception process, the controller changes a design of the answer image.

9. The cognitive function testing device according to claim 1, wherein
when the question word is switched in the answer reception process, the controller changes a display timing of the answer image.

10. The cognitive function testing device according to claim 1, wherein
the controller is configured to execute a training process between the output process and the answer reception process to reproduce content that applies a load on cognition of the examinee.

11. A non-transitory computer-readable storage medium storing a cognitive function testing program for causing a processor of a cognitive function testing device to execute
an output process of outputting a first word to be memorized to an examinee, and
an answer reception process of receiving, by causing a display device to display an answer image for receiving an answer for question words including the first word and a second word different from the first word sequentially, the answer from the examinee, the answer being as to whether the question word has been output in the output process, wherein
the answer reception process includes a process of changing a display mode of the answer image when the question word is switched.
